(19) United States
(12) Patent Application Publication
(10) Pub. No.: US 2018/0249734 A1
(43) Pub. Date: Sep. 6, 2018

(54) METHOD FOR PRODUCING MEAT CAPABLE OF REDUCING SATURATED FATTY ACID INTAKE

(71) Applicant: BIOBALANCE CO., LTD., Nagoya-shi, Aichi (JP)

(72) Inventor: Yoshio NAITO, Nagoya-shi, Aichi (JP)

(73) Assignee: BIOBALANCE CO., LTD., Nagoya-shi, Aichi (JP)

(21) Appl. No.: 15/756,493

(22) PCT Filed: Aug. 25, 2016
(86) PCT No.: PCT/JP2016/074747

§ 371 Date: Feb. 28, 2018

(30) Foreign Application Priority Data
Aug. 31, 2015 (JP) 2015-170466

(51) Int. Cl. A23K 10/18; A23K 50/10; A23L 13/00; C12N 1/20; C12R 1/25

(52) U.S. Cl. CPC A23K 10/18; A23K 50/10; A23L 13/00; A23V 2002/00; C12R 1/25; A23Y 2220/67; C12N 1/20

(57) ABSTRACT

A meat has a lowered amount of saturated fatty acid. By feeding a livestock animal with feed containing Lactobacillus plantarum BB-PLT (lactic acid bacterium BB-PLT strain) (NITE BP-02097), the saturated fatty acid contained in the meat from the livestock animal decreases and the monovalent unsaturated fatty acid contained in the meat from the livestock animal increases as a result of heating during cooking. The lactic acid bacterium strain Lactobacillus plantarum BB-PLT (NITE BP-02097) can be included in a livestock feed, and can be used in a method for raising a livestock animal for meat by feeding it to the livestock animal with the lactic acid bacterium. Edible meat can be obtained from a livestock animal raised by the method.

### CROSS-REFERENCE

This application claims the priority based on Japanese Patent Application No. 2015-170466 filed in Japan on 31 Aug. 2015. The contents described therein are incorporated herein by reference in their entirety. The contents described in patents, patent applications and literatures cited herein are incorporated herein by reference in their entirety.

### TECHNICAL FIELD

The present invention relates to a field to improve meat quality by lactic acid bacterium feed. Especially, the present invention relates to a method for providing meat containing less saturated fatty acid.

### BACKGROUND ART

Animal fat inherently contains more saturated fatty acid than that of plant fat. Therefore, meat has not been preferred because it may cause obesity or hyperlipidemia. However, meat is an important ingredient containing proteins and amino acids of good quality and is an essential food for dietary life.

### SUMMARY OF INVENTION

Accordingly, in order to provide soft meat containing lower amount of saturated fatty acid, the present inventor has studied with lactic acid bacteria, and consequently found that a livestock animal fed with feed containing Lactobacillus plantarum BB-PLT (lactic acid bacterium BB-PLT strain) (NITE BP-02097) gave meat which containing decreased amount of saturated fatty acid and increased amount of monovalent unsaturated fatty acid after heat cooked.

The present invention is based on the finding of Lactobacillus plantarum BB-PLT (lactic acid bacterium BB-PLT strain) (NITE BP-02097) and specifically relates to the following inventions:
(1) Lactobacillus plantarum BB-PLT (NITE BP-02097).
(2) Livestock feed comprising a lactic acid bacterium of (1).
(3) A method for raising a livestock animal for meat, comprising feeding the livestock animal with a lactic acid bacterium of (1).
(4) The method for raising of (3), wherein the livestock animal for meat gives meat whose containing amount of a saturated fatty acid contained decreases by heating.
(5) The method for raising of (4), wherein the saturated fatty acid is palmitic acid and/or stearic acid.
(6) The method for raising of any one of (3) to (5), wherein the livestock animal for meat gives meat whose containing amount of monovalent unsaturated fatty acid increases by heating.
(7) The method for raising of (6), wherein the monovalent unsaturated fatty acid is one or more monovalent unsaturated fatty acids selected from palmitoleic acid, oleic acid, and vaccenic acid.
(8) The method for raising of any one of (3) to (7), wherein the livestock animal for meat is cattle.
(9) Edible meat obtained from the livestock animal for meat raised by the method of any one of (3) to (8), or a processed product thereof.
(10) The edible meat of (9) or a processed product thereof, whose containing amount of saturated fatty acid decreases with heating.
(11) The edible meat of (10) or a processed product thereof, wherein the saturated fatty acid is palmitic acid and/or stearic acid.
(12) The edible meat of any one of (9) to (11) or a processed product thereof, whose containing amount of a monovalent unsaturated fatty acid increases with heating.
(13) The edible meat of (12) or a processed product thereof, wherein the monovalent unsaturated fatty acid is one or more monovalent unsaturated fatty acids selected from palmitoleic acid, oleic acid, and vaccenic acid.

Alternatively, the present invention relates to a feed composition, containing Lactobacillus plantarum BB-PLT (NITE BP-02097) as an active ingredient, which can give a livestock animal for meat containing a saturated fatty acid which decreases with heating and/or a monovalent unsaturated fatty acid which increases with heating.

Accordingly, in one aspect, the present invention relates to Lactobacillus plantarum BB-PLT (lactic acid bacterium BB-PLT strain) (NITE BP-02097) and livestock feed containing the same. The Lactobacillus plantarum BB-PLT (lactic acid bacterium BB-PLT strain) (NITE BP-02097) is a lactic acid bacterium isolated from the silage of Obihiro-city, Hokkaido, Japan, and was deposited under deposition No. NITE BP-02097 with National Institute for Technology and Evaluation (NITE) Patent Microorganisms Depositary (2-5-8, Kazusakamatari, Kisarazu-city, Chiba, Japan) on Aug. 10, 2015.

The livestock or the livestock animal described herein typically means an animal raised for the purpose of exploiting its products (milk, flesh, eggs, hair, skin, fur, labor, etc.). The livestock animal for meat described herein means an animal raised for the purpose of exploiting its product flesh. Examples of the livestock animal or the livestock animal for meat can include cattle, horses, water buffalos, deer, sheep, goats, alpacas, pigs, hogs, rabbits, chickens, quails, ostriches, turkeys, gooses, ducks, pheasants, and guinea fowls.

The “edible meat” described herein means flesh of the livestock animal intended to be eaten by humans and is preferably flesh to be eaten by cooking. The “processed product of the edible meat” is any processed product of the flesh of the livestock animal intended to be eaten by humans without particular limitations and includes processed products typically serving as flesh-derived preparation materials by production, such as ham, sausage, bacon, jerky, salami, and corn beef, as well as even prepared processed products such as hamburger, meatball, curry, meat sauce, and soup.

By eating the lactic acid bacterium BB-PLT strain described herein, the meat of the livestock for meat becomes that the saturated fatty acid contained therein decreases with heating. Examples of the saturated fatty acid can include lauric acid, myristic acid, palmitic acid, and stearic acid. Also, by eating the lactic acid bacterium BB-PLT strain described herein, and the meat of the livestock for meat becomes that the monovalent unsaturated fatty acid contained therein increases with heating. Examples of the monovalent unsaturated fatty acid can include α-linolenic acid, eicosapentaenoic acid, docosahexaenoic acid, linoleic acid, γ-linolenic acid, arachidonic acid, palmitoleic acid, oleic acid, and vaccenic acid.

### Advantageous Effects of Invention

By feeding a livestock animal with feed containing the lactic acid bacterium BB-PLT strain of the present invention, the saturated fatty acid contained in the edible meat decreases and the monovalent unsaturated fatty acid contained in the edible meat increases by cooking with heat. It is known that saturated fatty acids increase cholesterol levels, while unsaturated fatty acids decrease cholesterol levels. Therefore, feeding with the lactic acid bacterium BB-PLT strain described herein or the feed containing the same can provide healthier meat.

### BRIEF DESCRIPTION OF DRAWINGS

FIG. 1 is a graph of the result of saturated fatty acid analysis. Male fatten Limousin cattle (12 animals per group) were fed with 20 g/day/animal of a lactic acid bacterium formulation everyday, and its longissimus thoracis muscle at the 7th thoracic vertebrae was heat-treated at 95° C. The ordinate shows the amount of change (%) of saturated fatty acids after heating as compared with before heating. The black bar (left) shows a control group (non-administration of lactic acid bacterium formulation group). The amount of saturated fatty acids increased by 0.14%. The gray bar (right) shows an administered group with lactic acid bacterium formulation. The amount of saturated fatty acids decreased by 2.72%.

FIG. 2 is a graph of the results of monovalent unsaturated fatty acid analysis, which was conducted simultaneously with FIG. 1. The ordinate shows the amount of change (%) of monovalent unsaturated fatty acids. The black bar (left) shows the control group (non-administration group with of lactic acid bacterium formulation). The amount of monovalent unsaturated fatty acids increased by 0.74%. The gray bar (right) shows an administered group with the lactic acid bacterium formulation. The amount of monovalent unsaturated fatty acids increased by 3.32%.

FIG. 3 is a graph of results of analyzing the amount of change of each fatty acid in the experiment of FIG. 1. From the top to the bottom, the result for methyl cis-11-eicosenoate (C20: 1n9), vaccenic acid (C18: 1n7), oleic acid (C18: 1n9), palmitoleic acid (C16: 1n7), stearic acid (C18), palmitic acid (C16), pentadecylic acid (C15), and myristic acid (C14) are shown. The black bar (upper) shows the administration group with lactic acid bacterium formulation, and the gray bar (lower) shows the control group (non-administration group with lactic acid bacterium formulation).

### DESCRIPTION OF EMBODIMENTS

1. Culture of Lactic Acid Bacterium BB-PLT Strain

The bacterium can be cultured at a culture temperature of 15 to 40° C. (preferably 25 to 35° C.) and at culture pH of pH 3.5 to 9.0 (preferably pH 4.5 to 7.0) for a culture time of 6 to 30 hours using a medium suitable for lactic acid bacterium. As a specific example, the bacterium can be inoculated to a MRS liquid medium and cultured at 25 to 35° C. for 24 hours. For secondary culture, the culture solution can be further sprayed over sterilized bran and cultured at 25 to 35° C. for 3 days. In one aspect, the present invention relates to a use of the lactic acid bacterium BB-PLT strain for producing feed for rearing livestock for meat, wherein a saturated fatty acid contained in the meat from the reared livestock decreases by heating, and/or wherein a monovalent unsaturated fatty acid contained in the meat the reared livestock increases by heating. Alternatively, the present invention relates to the lactic acid bacterium BB-PLT strain for rearing livestock for meat whose saturated fatty acid decreases by heating, and/or whose monovalent unsaturated fatty acid increases by heating.

2. Feeding of Livestock Animal with Lactic Acid Bacterium BB-PLT Strain

In one aspect, the present invention relates to a method for raising a livestock animal, comprising feeding the livestock animal with the lactic acid bacterium BB-PLT strain (or livestock feed containing the lactic acid bacterium BB-PLT strain). In this method, the livestock animal is given the lactic acid bacterium BB-PLT strain with feed. The lactic acid bacterium BB-PLT strain can be appropriately provided, for example, in a solid (e.g., tablet, granule, or capsule), gel, or liquid form according to a feeding method. Preferably, the lactic acid bacterium BB-PLT strain is given thereto as a viable bacterium. The given amount can be determined depending on the body weight of the livestock animal. For example, for cattle, 10 to 100 g (preferably 40 g) per day can be given. The feeding period of the livestock animal with the feed of the present invention is not particularly limited, and can be changed according to the type of the livestock animal, and preferably is the whole raising period. However, the livestock animal may be fed with the feed of the present invention for only a portion (1 year, half year, 3 months, 1 month, etc.) of the raising period. Except for feeding with the feed of the present invention, the livestock animal can be raised by a conventional way.

Edible meat from a livestock animal for meat fed with the feed of the present invention and a processed product thereof can be obtained in the same way as used in livestock animals for meat fed with ordinary feed.

Hereinafter, the present invention will be described in more detail with reference to Examples. However, the present invention is not limited thereby. All literatures cited herein are incorporated herein by reference in their entirety.

### EXAMPLE 1

### Lactobacillus plantarum BB-PLT (Lactic Acid Bacterium BB-PLT Strain)

Lactobacillus plantarum BB-PLT (lactic acid bacterium BB-PLT strain) was isolated from the silage of Obihiro-city, Hokkaido, Japan. The lactic acid bacterium BB-PLT strain was deposited under deposition No. NITE BP-02097 (deposition date: Aug. 10, 2015) with National Institute for Technology and Evaluation (NITE) Patent Microorganisms Depositary (NPMD) (2-5-8, Kazusakamatari, Kisarazu-city, Chiba, Japan).

The mycological properties of the Lactobacillus plantarum BB-PLT (lactic acid bacterium BB-PLT strain) cultured using a MRS broth (manufactured by Oxoid SA) are as described below. As for sugar utilization, the culture was performed using a basal medium for carbohydrate fermentation tests (distilled water was added to 10.0 g of tryptone, 5.0 g of yeast extracts, and 0.06 g of bromocresol purple to adjust the total amount to 1000 ml, followed by pH adjustment to 6.8 and subsequent high-pressure sterilization at 121° C. for 15 minutes).

Cell morphology: bacillus

Spore formation: none

Motility: none

Gram staining: +

Sensitivity to oxygen: facultative anaerobic

Growth at 15° C.: +

Growth at 45° C.: −

Lactic acid fermentation: homo

Production of gas from glucose: −

Production of ammonia from arginine: −

Sodium hippurate hydrolyzability: −

Resistance to 4% sodium chloride: −

Sugar utilization: arabinose (−), xylose (−), rhamnose (−), ribose (+), glucose (+), mannose (+), fructose (+), galactose (+), sucrose (+), maltose (+), cellobiose (+), lactose (+), trehalose (+), melibiose (+), raffinose (+), melezitose (+), mannitol (+), sorbitol (+), esculin (+), salicin (+), amygdalin (+), sodium gluconate (+)

Produced Lactic Acid: DL-Lactic Acid

### EXAMPLE 2

### Preparation of Lactic Acid Bacterium BB-PLT Strain Probiotic

The bacterium was inoculated to a MRS liquid medium and primarily cultured at 25 to 35° C. for 24 hours. The culture solution was sprayed over sterilized bran and secondarily cultured at 25 to 35° C. for 3 days to obtain a lactic acid bacterium probiotic. The number of the bacterium contained in the obtained lactic acid bacterium probiotic was 3.0×10⁹ CFU/g.

### EXAMPLE 3

### Change in Fatty Acid Composition of Cattle by Feeding with Lactic Acid Bacterium Probiotic

(1) Feeding of Cattle with Lactic Acid Bacterium Probiotic and Sampling

Male fatten Limousin cattle (12 animals per group) were used. A lactic acid bacterium administration group was fed everyday with 20 g/day/animal of the lactic acid bacterium formulation as a mixture with ordinary feed. Immediately after slaughter, longissimus thoracis muscle at the 7th thoracic vertebrae was collected. The collected raw meat was vacuum-packed and transported in a refrigerated state of 5° C. or lower. For fatty acid composition analysis, unnecessary portions were removed, and the resultant was sliced as thin as possible perpendicularly to the muscle fiber orientation. The slices were cryopreserved at −30° C. until the analysis. The fatty acid composition analysis was conducted within 10 days after the start of the cryopreservation.

(2) Heat Treatment

Approximately 50 g of the sample was cut out into a cube to expose the muscle fiber. The cut piece was placed in a vinyl bag with its shape kept, which was placed in hot water at 95° C. until the core temperature reached 75° C., and taken out of the hot water, followed by cooling to room temperature.

(3) Fatty Acid Composition Analysis

Fatty acids were extracted with chloroform:methanol solution (V:V=2:1) and then analyzed by gas chromatography (Agilent Technologies 6890N).

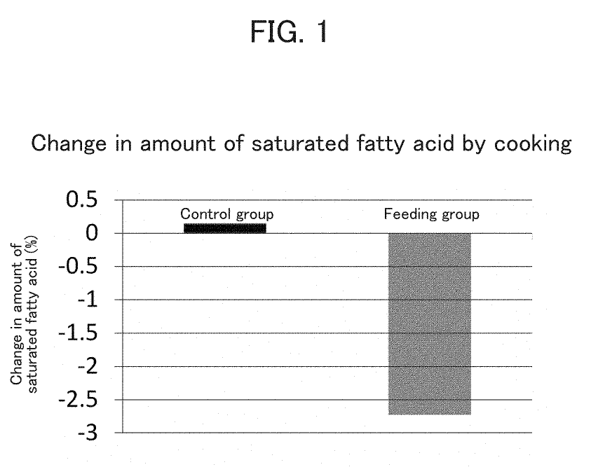

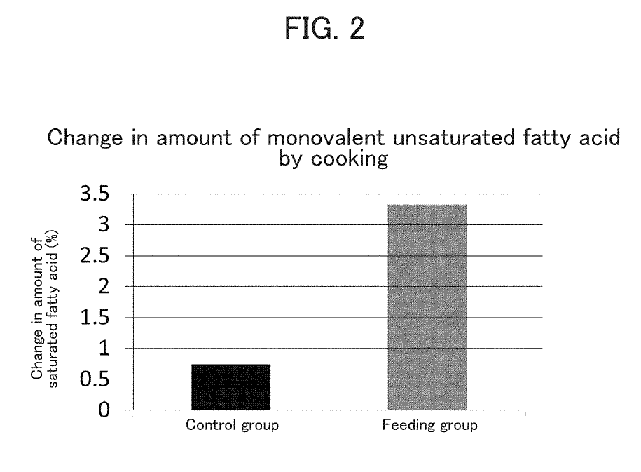

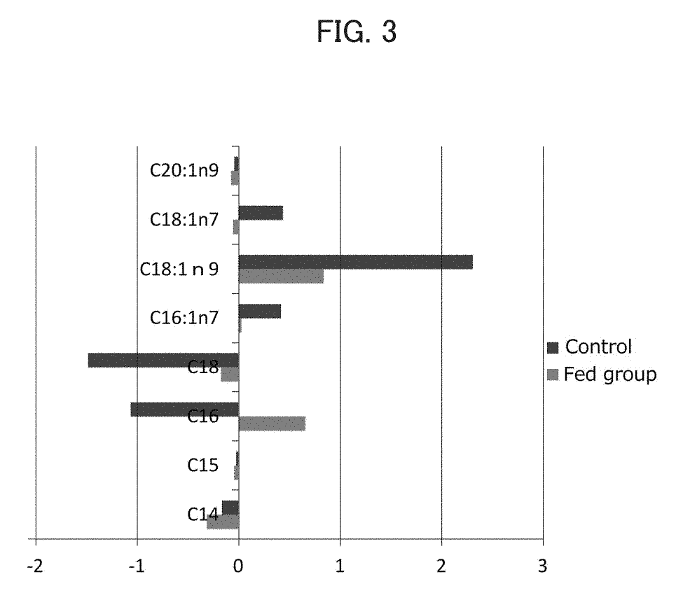

The results are shown in FIGS. 1 to 3. As a result, a fatty acid composition analysis after heat cooking raw meat obtained from the fatten cattle fed with the feed containing Lactobacillus plantarum BB-PLT has revealed that the saturated fatty acid decreased and the monovalent unsaturated fatty acid increased in the fed group. Each fatty acid component analysis showed that the amounts of palmitic acid (C16) and stearic acid (C18), which are the saturated fatty acids, were decreased, while the amounts of palmitoleic acid (C16: 1n7), oleic acid (C18: 1n9), and vaccenic acid (C18: 1n7), which are the monovalent unsaturated fatty acids, were increased. According to the above compositional change, the unsaturated fatty acid/saturated fatty acid ratio was also increased.

1. A strain of lactic acid bacterium, wherein the strain is Lactobacillus plantarum BB-PLT (NITE BP-02097).

2. A livestock feed comprising the lactic acid bacterium strain of claim 1.

3. A method for raising a livestock animal for meat, comprising feeding the livestock animal with the lactic acid bacterium strain of claim 1.

4. The method of claim 3, wherein the livestock animal for meat yields meat whose amount of a saturated fatty acid decreases by heating.

5. The method of claim 4, wherein the saturated fatty acid is selected from the group consisting of palmitic acid, stearic acid, and a combination thereof.

6. The method of claim 3, wherein the livestock animal for meat yields meat whose amount of a monovalent unsaturated fatty acid increases by heating.

7. The method of claim 6, wherein the monovalent unsaturated fatty acid is one or more monovalent unsaturated fatty acids selected from the group consisting of palmitoleic acid, oleic acid, and vaccenic acid.

8. The method of claim 3, wherein the livestock animal for meat is cattle.

9. An edible meat or a processed product thereof obtained from the livestock animal for meat raised by the method of claim 3.

10. The edible meat or the processed product thereof of claim 9, whose amount of a saturated fatty acid decreases by heating.

11. The edible meat or the processed product thereof of claim 10, wherein the saturated fatty acid is selected from the group consisting of palmitic acid, stearic acid, and a combination thereof.

12. The edible meat or the processed product thereof of claim 9, whose containing amount of a monovalent unsaturated fatty acid increases by heating.

13. The edible meat or the processed product thereof of claim 12, wherein the monovalent unsaturated fatty acid is one or more monovalent unsaturated fatty acids selected from the group consisting of palmitoleic acid, oleic acid, and vaccenic acid.
